(19) United States
(12) Patent Application Publication
(10) Pub. No.: US 2009/0191552 A1
(43) Pub. Date: Jul. 30, 2009

(54) Novel Human Acidic Mammalian Chitinase and Use Thereof

(76) Inventors: Kalpit A. Vora, Yardley, PA (US); Julie A. DeMartino, Westfield, NJ (US); John Stuart Mudgett, Basking Ridge, NJ (US); Gene Porter, Neptune, NJ (US); Gloria Wolfe, New York, NY (US)

Correspondence Address: MERCK AND CO., INC, P O BOX 2000, RAHWAY, NJ, 07065-0907, US

(21) Appl. No.: 12/227,719

(22) PCT Filed: Jun. 22, 2007
(86) PCT No.: PCT/US07/14772

§ 371 Date: Nov. 25, 2008

Related U.S. Application Data
(60) Provisional application No. 60/817,114, filed on Jun. 28, 2006.

(51) Int. Cl. C12Q 1/68; C12N 9/24; C07H 21/00; C12N 15/63; C12N 5/10

(52) U.S. Cl. 435/6; 435/200; 536/23.2; 435/320.1; 435/325

(57) ABSTRACT

Described herein is a variant human acidic mammalian chitinase (AMCase) enzyme having improved stability and an isolated nucleic acid sequence which encodes the variant. The invention also provides to nucleic acid constructs, vectors, and host cells comprising the nucleic acid sequences as well as methods for producing recombinant variant AMCase polypeptide and its use in screening assays to identify modulators.

### FIELD OF THE INVENTION

The present invention relates to a variant human acidic mammalian chitinase (AMCase) having improved stability and an isolated nucleic acid sequence which encodes the variant. The invention also relates to nucleic acid constructs, vectors, and host cells comprising the nucleic acid sequences. The invention further provides methods for producing the variant AMCase polypeptide and methods of using the variant enzyme to screen for AMCase modulators.

### BACKGROUND OF THE INVENTION

Chitin, a polymer of N-acetylglucosamine, is the second most abundant polysaccharide in nature. It is widely expressed as a surface component of the exoskeletons of many arthropods, of fungal cell walls, and of the microfilarial sheaths of parasitic nematodes. Generally speaking, chitin protects pathogens from harsh environmental conditions and host-mediated responses.

Chitinases (i.e., endo-β-3-1,4-acetylglucosanidases) are ubiquitous enzymes which cleave (hydrolyze) chitin. The innate immune response of many lower life forms, including plants, insects, and fish, to an infection by a chitin-containing pathogen includes chitinase induction. Family 18 chitinases define as group of enzyme which share a conserved catalytic site motif. Family 18 members hydrolyse β 1,4 linkages containing glutamic acid as a key catalytic site residue and proton donor.

Because chitin does not have a mammalian counterpart, it was generally assumed that mammals lack chitinolytic enzymes, and that the chitinase genes found in the human genome merely represent relics of evolution. However, recent studies have identified chitinase and chitinase-like enzymes in both rodents and human subjects. The lack of endogenous mammalian chitin suggests that other, unidentified endogenous substrates, or other enzymatic activities remain to be characterized.

Acidic mammalian chitinases (AMCases) represent a unique subset of mammalian enzymes because they hydrolyze chitin and chitin-like substrates with an acidic pH optimum. AMCase is known to be predominantly expressed in the gastrointestinal tract and stomach where it is thought to play a role as antiparasitic defense mechanism. It may also play a role in the normal digestive process. AMCase is expressed to a lesser extent in the lung. During acute asthmatic attacks exhaled airway vapor condensates are known to be acidified, and the upregulation of AMCase expression observed in murine models of asthma suggests that chitinases are involved in allergic immune responses. However, there remains a need to understand the role of chitinases the pathogenesis of asthma, and to establish screening assays capable of identifying potential therapeutics which are capable of modulating molecular targets implicated in mammalian disease processes.

### SUMMARY OF THE INVENTION

The present invention relates to a novel human acidic mammalian chitinase (hAMCase) isoform (allelic variant) and a nucleic acid molecule (a cDNA) encoding the variant enzyme. The disclosed allelic variant differs by three amino acids from the reference sequence published in GenBank as AF 290004 (wild-type human AMCase) (SEQ ID NO: 4) (also referred to herein as the Boot Sequence) (J. Biol. Chem. 276(9):6670-6678 (2001). The three amino acid substitutions present in the disclosed variant of hAMCase appear to lend stability to the recombinantly expressed protein, which in turn allows for more efficient production of active enzyme. Therefore, the variant facilitates the development and use of high through put screening assays designed to identify and study modulators of the enzyme's activity. The knowledge that AMCase expression/activity is associated with inducing the symptoms and/or complications of asthma renders AMCase sequences useful in methods for identifying agents which are capable of modulating AMCase activity and which therefore represent potential therapeutic agents.

One aspect of the present invention provides an isolated human acidic mammalian chitinase (AMCase) variant (or isoform) consisting of the amino acid sequence set forth in SEQ ID NO: 2. Importantly, the disclosed human AMCase variant has improved stability relative to wild-type human AMCase. In one embodiment of this aspect of the invention the disclosed AMCase variant comprises a substitution at a position corresponding to one or more of residues N45, D47, and R61 SEQ ID NO: 4, which sets forth the reference amino acid sequence of what is referred to herein as wild-type AMCase. In an alternative embodiment the invention provides a human AMCase variant with improved stability relative to wild-type AMCase wherein the variant comprises substitutions at all three positions corresponding to N45, D47, and R61, as exemplified by the amino acid sequence set forth in SEQ ID NO: 2 which comprises three substitutions (D45N, N47D, and M61R) substitutions relative to the amino acid sequence of the reference AMCase protein.

A second aspect of the invention provides a nucleic acid molecule which encodes either the full-length variant enzyme or a fragment of the disclosed AMCase variant. One embodiment of this aspect of the invention is provided by SEQ ID NO: 1, which provides a cDNA which encodes the full-length variant AMCase. One of skill in the art will readily acknowledge that due to degeneracy of the genetic code, alternative nucleic sequences capable of coding for the amino acid sequence set forth in SEQ ID NO: 2 can be utilized to express the variant isoforms in suitable transformants. Accordingly, the scope of the invention includes all nucleic acid molecules which include alternative nucleotide sequences which encode the disclosed variant enzyme.

Other aspects of the invention provide expression vectors comprising nucleic acids which encode the variant AMCase, and host cells transformed with the expression vectors of the invention. The transformed host cells disclosed herein can be used in the context of a method suitable for the production of the disclosed variant AMCase enzyme, which can be subsequently purified from tissue culture media and used to establish an enzymatic activity assay designed to identify and characterized compounds that are capable of modulating AMCase's enzymatic activity.

For example, the disclosed AMCase variant can be employed in a high throughput screening assay to determine whether a test compound is capable of modulating the enzymatic (e.g. chitinolytic) activity or level of AMCase. Accordingly, the sequences, vectors and host cells of the invention can be used in a method which is based on the following steps: 1) transfecting producer host cells with an expression vector comprising a nucleic acid encoding a variant AMCase wherein the variant consists of the amino acid sequence set forth in SEQ IN NO: 2; 2) culturing the transformed producer cells under conditions suitable to express the variant AMCase; 3) purifying variant AMCase from the culture medium; 4) exposing the variant AMCase to a test compound in the presence of a detectably chitinase substrate; 5) measuring the chitinolytic activity of the variant AMcase; and 6) comparing the chitinolytic activity of the variant AMcase observed in the absence of the test compound with the chitinolytic activity observed in the presence of the compound; wherein if the level of chitinolytic activity in the presence of the test compound differs from the amount of activity in the absence of the test compound, then the test compound is capable of modulating AMCase activity.

Other embodiments, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description. The examples provided in this disclosure are not intended to limit the scope of the claimed invention.

### BRIEF DESCRIPTION OF THE DRAWINGS

FIG. 1 provides the nucleotide sequence (SEQ ID NO: 1) of a cDNA which encodes the AMCase allelic variant of the invention.

FIG. 2 provides the nucleotide sequence of a 1229 base pair nucleic acid cloned from human stomach DNA (SEQ ID NO: 5) (referred to as “hAMCase (stom) 5′ end).

FIG. 3 provides the nucleotide sequence of a 561 base pair fragment cloned from human total lung RNA (SEQ ID NO: 6. The bolded and italicized nucleotides are different from the nucleotides reported in the wild type Boot sequence.

FIG. 4 provides an alignment of SEQ ID NO: 6 (hAMCase 5′ end MRL) with the 5′ end of wild-type human AMCase (SEQ ID NO: 3) (the Boot sequence).

FIG. 5 provides the amino acid sequence (SEQ ID NO: 2) of the human AMCase (hAMCase) variant encoded by SEQ ID NO: 1, and a graphic representation of the location of various protein domains.

FIGS. 6A and 6B provide an alignment comparing the nucleotide sequence of SEQ ID NO: 1 cDNA for variant hAMCase (MRL sequence) with the nucleotide sequence of the wild-type AMCase coding sequence of GenBank Accession AF 290004 (SEQ ID NO: 3).

FIG. 7 provides an alignment comparing the amino acid sequence of SEQ ID NO: 2 (h-AMCase) (MRL sequence) with the amino acid sequence (SEQ ID NO: 4) (Boot sequence) of the wild-type AMCase product of SEQ ID NO: 3.

FIG. 8 is a graphic representation comparing the enzymatic activity of the hAMCase variant of the invention compared with the enzymatic activity of the wild-type AMCase (Boot AMCase).

FIG. 9 provides a graphic representation of the pH profile and reaction max of the variant and wild-type AMCases.

FIG. 10 provides a graphic representation of the effect of pH on the Allosamidin IC50 value of the variant and wild-type AMCases.

FIG. 11 provides an alleleic discrimination plot resulting from a single nucleotide polymorphism (SNP) assay.

FIG. 12 provides the Smith-Waterman sequence alignment data for hAMCase and its most homologous protein Ym1 (SEQ ID NO: 7) (PDB code: 1E9L).

FIGS. 13A and 13B provide an alignment comparing the amino acid sequences of Elias (NM_{—}021797) (SEQ ID NO: 8), Saito (AB025008) (SEQ ID NO: 9), Boot (AF290004) (SEQ ID NO: 4), the MRL AMCase variant (SEQ ID NO: 2), and the translation of NM201653 (SEQ ID NO: 10) which represents a hAMCase consensus sequence.

### DETAILED DESCRIPTION OF THE INVENTION

The patent and scientific literature referred to in this disclosure is cited to establish knowledge that is available to those of skill in the art. The citations referred to herein, including issued U.S. patents, published patent applications (U.S. and foreign), references including GenBank database sequences, cited herein are incorporated by reference to the same extent as if each was individually and specifically incorporated by reference. The references cited throughout the present disclosure are not admitted to be prior art to the claimed invention.

Unless defined otherwise, technical and scientific terms used herein have the meanings commonly understood by one of ordinary skill in the art to which the present invention pertains. One skilled in the art will recognize other methods and materials similar or equivalent to those described herein, which can be used in the practice of the present teaching. It is to be understood, that the teachings presented herein are not intended to limit the methodology or processes described herein. For purposes of the present invention, the following terms are defined below.

“Chitinase,” as used herein, refers to a family of polypeptides comprising mammalian chitinases. A chitinase of the present invention demonstrates detectable chitinase activity, in that it specifically cleaves chitin in an endochitinase manner.

An “AMCase modulator,” as the term is used herein, includes a molecule compound (or agent), that modulates the enzymatic activity of AMCase. Such modulators includes, but are not limited to, small molecules and chemical compounds that increase or inhibit the level of AMCase activity in a cell or tissue compared with the level of AMCase activity in the cell or tissue in the absence of the modulator, or in an otherwise identical cell or tissue, in the absence of the modulator. Agents which decrease the enzymatic activity of AMCase represent a subset of modulators which represent potential therapeutic agents because of their ability to inhibit AMCase.

The term “polypeptide” or “protein” as used herein refers to a compound made up of a single chain of amino acid residues linked by peptide bonds.

As used herein the term “nucleic acid” or “nucleic acid molecule” includes RNA, DNA, and cDNA molecules. It will be understood that, as a result of the degeneracy of the genetic code, a multitude of nucleotide sequences encoding a given protein such as AMCase may be produced. The present invention contemplates every possible variant nucleotide sequence, encoding AMCase, all of which are possible given the degeneracy of the genetic code.

As used herein, the term “gene” means the segment of DNA involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge

As used herein an “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

As used herein, the term “plasmid” refers to a circular double-stranded (ds) DNA construct used as a cloning vector, and which forms an extrachromosomal self-replicating genetic element in many bacteria and some eukaryotes.

As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

As used herein, the term “selectable marker-encoding nucleotide sequence” refers to a nucleotide sequence which is capable of expression in cells and where expression of the selectable marker confers to cells containing the expressed gene the ability to grow in the presence of a corresponding selective agent, or under corresponding selective growth conditions.

A nucleic acid is “operably linked” when it is placed into a functional relationship with another nucleic acid sequence. For example, DNA encoding a secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors, linkers or primers for PCR are used in accordance with conventional practice.

The term “introduced” in the context of inserting a nucleic acid sequence into a cell, means “transfection” or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

By the term “host cell” is meant a cell that contains a vector and supports the replication, and/or transcription or transcription and translation (expression) of the expression construct. Host cells for use in the present invention can be prokaryotic cells, such as E. coli, or eukaryotic cells such as yeast, plant, insect, amphibian, or mammalian cells. In general, host cells are filamentous fungi.

As used herein, the term “expression” refers to the process by which a polypeptide is produced based on the nucleic acid sequence of a gene. The process includes both transcription and translation.

As used herein, the terms “active” and “biologically active” refer to a biological activity associated with a particular protein and are used interchangeably herein. For example, the enzymatic activity associated with a chitinase is hydrolysis and, thus, an active chitinase has hydrolytic activity. It follows that the biological activity of a given protein refers to any biological activity typically attributed to that protein by those of skill in the art.

As used herein the term “variant” generally means a protein that is related to a parental protein (e.g., the wild-type protein) by addition of one or more amino acids to either or both the C- and N-terminal end, substitution of one or more amino acids at one or a number of different sites in the amino acid sequence, or deletion of one or more amino acids at either or both ends of the protein or at one or more sites in the amino acid sequence. As used herein, “variants”, includes polynucleotides or polypeptides containing one or more deletions, insertions or substitutions, as long as the variant retains substantially the same activity of the wild-type polynucleotide or polypeptide. With regard to polypeptides, deletion variants are contemplated to include fragments lacking portions of the polypeptide not essential for biological activity. Substitution variants include polypeptides consisting of one or more nucleic acids which differ from the nucleic acid which occupies the corresponding residue position in the wild-type AMCase polypeptide.

The term “variant acidic mammalian chitinase gene” or “variant AMCase” means, respectively, that the nucleic acid sequence of the AMCase reference gene has been altered by removing, adding, and/or manipulating the coding sequence of the gene, or that the amino acid sequence of the expressed protein has been modified consistent with the invention described herein.

As used herein the term “allosamidin” refers to a pseudo-trisaccharide with the chemical name (3aR,4R,5R,6S,6aS)-2-(dimethylamino)-3a,5,6,6a-tetrahydro-4-hydroxy-6-(hydroxymethyl)-4H-cyclopentoxazol-5-yl 2-(acetylamino)-4-O-[2-(acetylamino)-2-deoxy-β-D-allopyranosyl]-2-deoxy-β-D-allopyranoside and formula C₂₅H₄₂N₄O₁₄ which is a pan-family 18 chitinase inhibitor.

As used herein the term “stability” refers the ability to retain enzymatic activity during the purification process and/or to the observation of no loss of enzymatic activity upon repeated freeze thawing cycles.

As used herein “improved production” refers to increased yield of enzyme obtained from transfected supernatants of host cells (e.g., HEK293 cells) transfected with similar amounts of DNA.

The mammalian chitinase field has been hampered by an inability, using constructs encoding the published cDNA sequence of human AMCase (hAMCase), to express sufficient recombinant enzyme for characterization, structural studies or HTS development. This invention details the identification of a novel, naturally occurring isoform/allelic variant of hAMCase which differs in sequence by three amino acids from the reference sequence published as GenBank AF 290004 (SEQ ID NO: 4). As shown herein the three amino acid substitutions present in the disclosed variant of hAMCase confers stability to the recombinantly expressed protein, which in turn has the effect of allowing for more efficient production of active enzyme. Moreover, the much greater quantities of recombinant active enzyme produced by host cells expressing the variant sequence makes structural studies of enzyme/inhibitor complexes more feasible which in turn may expedite, through rationale drug design, the identification of chitinase inhibitors characterized by enhanced potency and selectivity.

Chitin is a glycopolymer of B(1-4 linked N-acetyl-D-glucosamine units. Chitin may also contain glucosamine units in different proportions. The chain length of N-acetylglucosamine polymers have been reported to range from 100 to approximately 8000 units. Typically, the polymers assemble laterally to form microfibrils, stabilized by strong hydrogen bonds between the amine group of sugar in one chain and the carbonyl group of sugar in an adjoining chain. To date, three crystallographic forms of chitin have been reported.

Chitonases are widespread in nature, and all chitin-containing organisms are presumed to require enzyme systems that allow them to degrade chitin polymers as a prerequisite for morphogenesis (i.e., essential modification of their shape). Several enzymatic activities can be distinguished: 1) B-hexosaminidases that are capable of removing the terminal N-acetylglucosamine moiety from the non-reducing end of the polysaccharide; 2) lysozymes with a broad specificity, such as egg white lysozyme, are capable of leaving the chitin glycopolymer; 3) exochitinases which cleave diacetylchiobiose units from the nonreducing end of the polysaccharide; and 4) specific endochitinases which cleave glycoside linkages randomly along the chitin chain, thereby giving rise to diacetylchitobiose as a major product together with some triacetylchitotriose. Typically, the term chitinase is used to refer to enzymes with either an exo- and endochitinase activity. As used herein, the term chitinase refers to a family of polypeptides which specifically cleave chitin in an endochitinase manner.

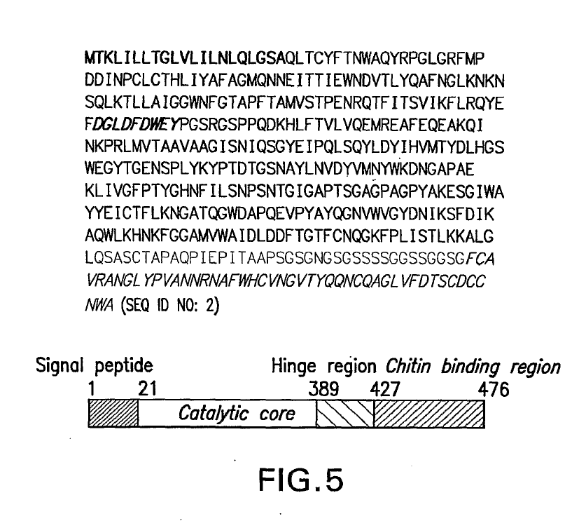

Chitinases constitute families 18 and 19 of the glycosylhydrolases. Recent data suggesting that AMCase and other Family 18 chitonases play a key role in the pathophysiology of inflammatory disorders, including lung inflammation and asthma support the assertion that Family 18 chitonases represent validated drug targets. Family classifications are based on amino acid sequence similarity. The typical structure of a chitinase consists of a signal peptide, a glycohydrolase domain and a chitin-binding domain. AMCase contain a N-terminal catalytic core domain, and a C-terminal chitin binding domain separated by a hinge region (see FIG. 5). Thus far, one or more of these characteristic features can be found in all of the identified and characterized mammalian chitinases. There is considerable homology in the putative active site regions of Family 18 members and the reaction mechanism is believed to be general acid-base catalysis.

The catalytic domain of family 18 chitinases has a (αβ)₈ (TIM barrel) fold with conserved DXDXE (SEQ ID NO: 23) catalytic motif that spans strand 4 of the TIM barrel and includes the glutamate that acts as the catalytic acid. However, many family members have been found lacking chitinolytic activity because of mutations in the highly conserved catalytic domain of these proteins. The amino acid sequence of AMCase includes this conserved region (133-FDGLDFDWEYPG-143) (SEQ ID NO: 11) as reported for many chitinases that show catalytic activity. When the catalytic site was mutated by replacing 2 amino acids using point mutations in murine chitotriosidase (FDGLNLDWQFPG) (SEQ ID NO: 12) the recombinant protein was no longer active. Chilectins like YKL-40 lack the active site glutamic acid (FDGLDLAWLYPG) (SEQ ID NO: 13) which is replaced by glutamine and hence lack enzyme activity. The active site grooves of these chitinases are lined with aromatic amino acids that contribute to polysaccharide substrate binding.

AMCase has been implicated as a potential drug target for airway disease like asthma/COPD and inflammatory diseases of the GI tract including IBD/Crohns. Therefore, the invention provides a novel allelic variant of acidic mammalian chitinase (AMCase) and its DNA, which is useful as a druggable target for the development of compounds capable of inhibiting the pathophysiological processes implicated in airway disease like asthma/COPD and inflammatory diseases of the GI tract including IBD/Crohns.

AMCase has been reported to be highly expressed in the lungs of mice sensitized to ovalablumin, and it has been implicated as playing a role in the pathophysiology of bronchial asthma in murine models. A gene expression study published by Zimmermann et al. has observed that AMCase expression is directly correlated with an asthmatic phenotype (Zimmermann et al., J. Immunol. 172:1815-1824 (2004). Inhibition of AMCase has been reported to lead to abrogated T-helper cell type 2-mediated inflammation, to reduce bronchial hyperactivity, and to lower eosinophil counts. In humans, AMCase has been observed to be highly expressed in the lungs of asthmatic patients, but not in the lungs of healthy individuals (Zhu, Z., et al. Science 304:1678-1682 (2004). In addition, the recent description of a polymorphism in AMCase that is associated with lower bronchodilator responsivemess in Puerto Rican asthmatics further supports the position that AMCase plays a role in the pathogenesis of asthma (Siebold, M. A., et al., Proc. Am. Thor. Soc., 2:A79 (2005).

Using the sequences provided herein, a skilled artisan can utilize well-known methods to produce and purify the variant enzyme for use in a HTS assay to identify AMCase modulators. The expressed protein can be purified from the supernatants or from cell lysates using standard protein purification protocols. A skilled artisan will readily acknowledge that an AMCase polypeptide can be purified by chromatographic, electrophoretic or centrifugation techniques. A representative, non-limiting purification protocol is described in the examples at the end of this disclosure.

A nucleotide sequence encoding a human AMCase polypeptide (e.g., a cDNA) having the amino acid sequence of the disclosed variant may be inserted in an expression vector for subsequent protein production by a suitable host cell. Any of a number of expression vectors could by used to produce the variant, including plasmid or viral vectors. For example, retroviral, adenoviral, or adeno-associated viral vectors could be used. Typically, a recombinant expression vector includes other known genetic elements (e.g., regulatory elements), that are either desirable or necessary to direct the efficient expression of the nucleic acid in a specific host cell, operatively linked to a coding sequence.

At a minimum expression vectors typically comprise a promoter and any necessary enhancer sequences to achieve transcription of the coding sequence. As used herein, a nucleotide sequence is “operatively linked” to another nucleotide sequence when it is placed in a functional relationship with the other sequence. In the case of a promoter element and a coding sequence this typically means that the DNA sequences are contiguous such that the promoter can function to initiate transcription of the coding sequence.

Expression vectors may optionally include a reporter gene operably linked to the promoter element. Suitable genes may encode, for example, luciferase, B-galactosidase, chloramphenical acetyltransferase, B-glucoronidase, alkaline phosphatase, green fluorescent protein, or other reporter gene products known to the art.

For example, an AMCase polypeptide can be expressed by cloning a nucleotide sequence encoding a polypeptide consisting of the variant AMCase amino acid sequence set forth in SEQ ID NO: 2 into the multiple cloning region of the expression vector p3Xflag-CMV13 (sigma-E4776) or any other eukaryotic expression vectors not limited to (pcDNA, pCImamalian expression vector, pIRES etc) available commercially or of propriety origin. Such a vector contains a leader sequence 5′ of the multiple cloning sequences to facilitate excretion of the translated protein into the media. The vector also contains 3 adjacent tagged epitopes (e.g. Flag, His etc) downstream of the multiple cloning regions to encode a c-terminal tag peptide facilitating its purification. The vector also contains the selectable marker, aminoglycoside phosphotransferase II gene (neo) which confers resistance to G418 and allows for the production of stable transfectants. Transcription of each cDNA is driven by the promoter-regulatory region of the human cytomegalovirus or any other suitable promoter and followed by the polyadenylation/transcription termination signal of eukaryotic origin for example the human growth hormone gene (hGH PolyA).

Alternatively, separate constructs can also be constructed without the tag or cleavable tag which have cleavable sequence (like Factor Xa) added downstream of the respective cDNA and just upstream of the tag epitope. This will allow for the removal of the tag after translation and purification.

A wide variety of host cells can be used to produce the desired quantity of variant AMCase protein. Such cells include, but are not limited to, prokaryotic and eukaryotic cells, including bacterial or mammalian host cells well-known in the art. Exemplary host cells include, HRK293 A cells, Chinese hamster ovary cells (CHO), COS cells, yeast, or bacteria belonging to the genus E. coli or Bacillus.

Numerous methods are available to introduce an expression vector into a host cell, including mechanical methods, chemical methods, lipophilic methods or electroporation. Use of calcium phosphate or DEAE-Dextran are representative, non-limiting examples of available chemical methods of introducing an expression vector into a host cell. Use of gene gun and microinjection are examples of mechanical methods. Lipophilic methods include use of liposomes and other cationic agents for lipid-mediated transfection.

Production of a variant enzyme is typically achieved by preparing a DNA sequence which encodes the variant protein, constructing a suitable expression vector based on the host cell of choice, transforming the host cell according to a protocol that will result in transient or stable transfection, and culturing the transformant under suitable conditions to express and produce the recombinant enzyme. As shown herein the variant AMCase enzyme of the invention provides a polypeptide comprising altered amino acid sequences in comparison with the amino acid sequence of the wild-type enzyme and while the variant enzyme retains the characteristic chitinolytic nature of the reference enzyme it produces a more stable recombinant protein.

As shown herein, the disclosed nucleic and amino acid sequences of the AMCase variant can be used to establish a HTS assay designed to identify agents which are capable of modulating AMCases enzymatic activity. Because the endogenous human (or mammalian) substrate for AMCase has not yet been identified, in vitro enzymatic activity assays are typically performed with a chitin-like substrate such as chito-biose or triose linked to 4-methylubelliferyl beta-D-N,N′,N″-triacetylchitotriose.

Numerous artificial chitinase substrates can be used to determine the chitinolytic activity of an AMCase or AMCase variant enzyme including, but not limited to: 4-methylubelliferyl beta-D-N,N′,N″triacetylchitotriose, polymers of chitin or their derivatives, N^{I},N^{II},N^{III},N^{IV}-Tetra-acetyl-NV-dimethylaminophenylazophenyl-thioureido-β-chitopentaosyl-pyroglutamyl amidoethylnaphtalene sulfonic acid. Two substrates that have been used routinely for assay are 4-methylubelliferyl beta-D-N,N′,N″-triacetylchitotriose, and 4-methylubelliferyl beta-D-N,N′-Diacetylchitobiose. Besides these polymers of chitin or their derivatives, NI, NII, NIII, NIV-Tetra-acetyl-NV-dimethylaminophenylazophenyl-thioureido-chitopentaosyl-pyroglutamyl amidoethylnaphtalene sulfonic acid could be used to evaluate the enzymatic activity.

Any other substrate for chitinases that allows easy detection of enzymatic activity could be used including fluorescent substrates, colored dyes and turbid substrates that become clear upon subjected to chitinase activity. These may include colloidal chitin, glycol chitin, 3-4 dinitrophenyl tetra-N-acetyl chitotrioside. Any type of solid or liquid media that will support growth and reproduction of recombinant or natural chitinase secreting, bacteria, yeast or mammalian cell also amounts to measuring the enzyme activity.

In practice, a wide variety of in vitro assay formats using an isolated AMCase polypeptide can be used to determine whether a test agent modulates the activity of the human AMCase enzyme. For example, the amount of reactants remaining and/or products produced in reactions catalyzed by AMCase can be quantified. It is well-known that AMCase catalyzes the conversion of chitin to N-acetyl-D-glucosamine. A nonlimiting example of a suitable assay design would involve monitoring the conversion of chitin or a chitin-like compound to N-acetyl-D-glucosamine. The enzyme activity could also be monitored by estimating the amount of substrate left or the products formed. A non-limiting example of substrate being chitin or its derivatives and N-acetyl-D-glucosamine of the product formed due to chitinase activity. Additionally, the skilled artisan would appreciate, once armed with the teachings of the present invention, that inhibition of a chitinase enzyme includes inhibition of the chitinolytic activity in a cell.

Possible reactions which could be quantified include, without limitation, the release of 4-methylumbelliferyl β-D-N,N′-diacetylchitobiose or 4-methylumbelliferyl β-D-N,N″-triacetylchitotriose; or the release of p-nitrophenyl from p-nitrophenyl β-D-N,N′,N″-triacetylchitotriose. In practice, the amount of chitin remaining after AMCase is contacted with the test agent as a function of time is determined. Similarly, the amount of N-acetyl-D-glucosamine or 4-methylumbelliferyl or p-nitrophenyl produced after the chitinase is contacted with the test agent, in the presence of chitin, as a function of time could also be monitored. A skilled artisan can easily identify various assays that can be used to quantify one of these products and/or reactants.

Methods of quantitating chitin are known to the art, including the use of various immunoassays, such as enzyme-linked immunoassays. Alternatively, a colorimetric assay can be used to determine the quantity of N-acetyl-D-glucosamine, as described in Reissig, J. L., J. Biol. Chem. 217:959-966 (1955). Well-known chromatographic methods, including high performance liquid chromatography can be employed to determine the amount of glucosamine. Fluorometric assays could be used to determine the amount of 4-methylumbelliferyl or p-nitrophenyl as described, for example in U.S. Pat. No. 5,561,051. Alternatively, fluorophore-assisted carbohydrate electrophoresis could also be used to monitor an AMCase catalyzed reaction.

One of skill in the art would readily appreciate, based on the disclosure provided herein, that a AMCase modulators include chemical compounds that inhibit the activity of a chitinase. Chitinase inhibitors are well known in the art, and some of the key critical elements of one class of chitinase-like molecule inhibitors have been defined (Spindler and Spindler-Barth, 1999, Chitin and Chitinases, Birkhauser Verlag Basel, Switzerland). Additionally, a chitinase-like molecule inhibitor encompasses a chemically modified compound, and derivatives, as is well known to one of skill in the chemical arts.

The skilled artisan would appreciate that AMCase inhibitors include well-known agents such as, but not limited to, allosamidin (Allosamidine, Carbohydrate Chemistry Industrial Research Limited, Lower Hutt, New Zealand, and Eli Lilly and Co., Greenfield, Ind.) and its derivatives (see, e.g., U.S. Pat. No. 5,413,991), glucoallosamidin A, glucoallosamidin B, methyl-N-demethylallosamidin (Nishimoto et al., 1991, J. Antibiotics 44:716-722) demethylallosamidin (U.S. Pat. No. 5,070,191), and didemthylallosamidin (Zhou et al., 1993, J. Antibiotics 46:1582-1588). Such agents provide the skilled artisan with a positive control which can be used to validate a HTS assay designed to identify AMCase modulators that are of potential therapeutic value because of their ability to inhibit AMCase. Alternatively, screening assays could also be validated using other known inhibitors of chitinase enzymatic activity, including, inter alia, allosamidin, 1,10-phenanthroline, glucoallosamidin A, glucoallosamidin B, methyl-N-demethylallosamidin, demethylallosamidin, didemthylallosamidin.

Further methods of identifying and producing potential AMCase are well known to those of ordinary skill in the art, including, but not limited, obtaining molecular agents from a naturally occurring source (i.e., Streptomyces sp., Pseudomonas sp., Stylotella aurantium). Alternatively, potential AMCase modulators can be synthesized chemically. Compositions and methods for chemically synthesizing potential modulators or for obtaining them from natural sources are well known in the art and are described in, among others, Yamada et al., U.S. Pat. Nos. 5,413,991, and 5,070,191.

Examples are provided to further illustrate different features of the present invention. The embodiments provided identify useful reagents and methodologies that are useful for practicing the invention. These examples do not limit the claimed invention.

Any suitable materials and/or methods known to those of skill can be utilized in carrying out the present invention; however, preferred materials and/or methods are described. Materials, reagents and the like to which reference is made in the following examples are obtainable from commercial sources, unless otherwise noted.

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2^{nd} Edition, Cold Spring Harbor Laboratory Press, 1989.

### EXAMPLES

### Example 1

### Cloning hAMCase Variant cDNA

Cloning of Partial Human AMCase Variant cDNA from Stomach

Human AMCase cDNA was cloned by PCR amplification of Poly A+ RNA from using Quick-Clone cDNA (BD Biosciences. Cat. # 7126-1). The source of the Poly A+ RNA was normal stomachs pooled from 10 male/female Caucasians, ages 25-53 and the cause of death was trauma. We used the following PCR primers: ACMF primer 5′-gccaccatgacaaagcttattctcctcacaggtcttg-3′ (SEQ ID NO: 14) and ACMR primer 5′-ttatgcccagttgcagcaatcacagotggtgtcgaag (SEQ ID NO: 15). The primers were made according to the published sequence for wild-type AMCase (SEQ ID NO: 3) (accession number AF 290004).

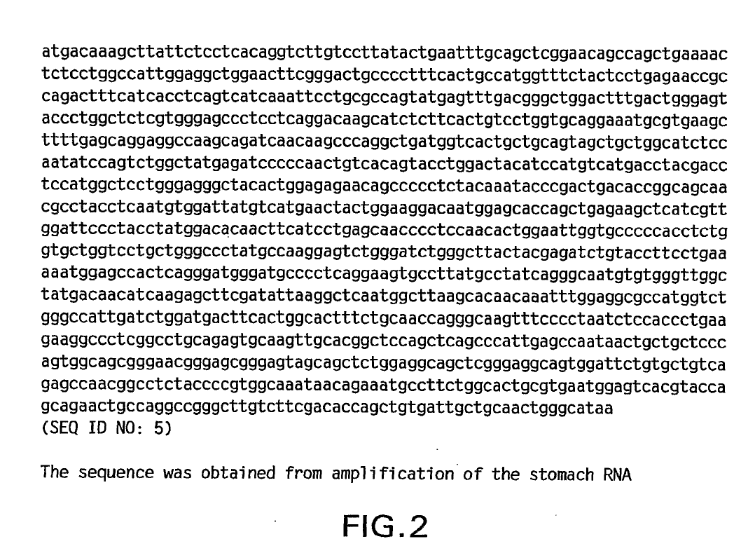

Two PCR amplification reactions were performed with Pfiu Turbo® DNA Polymerase (Stratagene Cat #600153) under the following conditions: 95 C for 2 minutes, and 30 cycles of 95° C. for 45 seconds, 55° C. for 45 seconds and 70° C. for 2 minutes. The reaction was terminated with one 10 minute cycle at 70° C. The PCR reactions were pooled and cloned in pPCR-Script Amp SK (+) vector (Stratagene Cat. # 211188), and sequenced several clones. A cDNA consisting of 1229 bp (FIG. 2) was obtained. A 1229 bp partial AMCase cDNA sequence (SEQ ID NO: 5) was the predominant spliced form isolated from the Stomach PCR reaction. When we aligned our sequence with the published sequence (accession number AF 290004) we observed a gap of 202 bp (exon 4) in the 5′ end. The rest of the sequence was identical to the published sequence.

Cloning the 5′ end of the Human AMCase Variant cDNA from Lung

The 5′ end of human AMCase was amplified by RT/PCR of human lung total RNA (BD Biosciences Cat. #64092-1). The source of the total RNA was normal lungs pooled from 3 male/female Caucasians, ages 15-40 and the cause of death was sudden death. The following PCR primers were used: ACMF primer 5′-gccaccatgacaaagcttattctcctcacaggtcttg-3′ (SEQ ID NO: 14) and ACMR2 primer 5′-ggagatgccagcagctactgcagc-3′ (SEQ ID NO: 16) made according to the published sequence (accession number AF 290004).

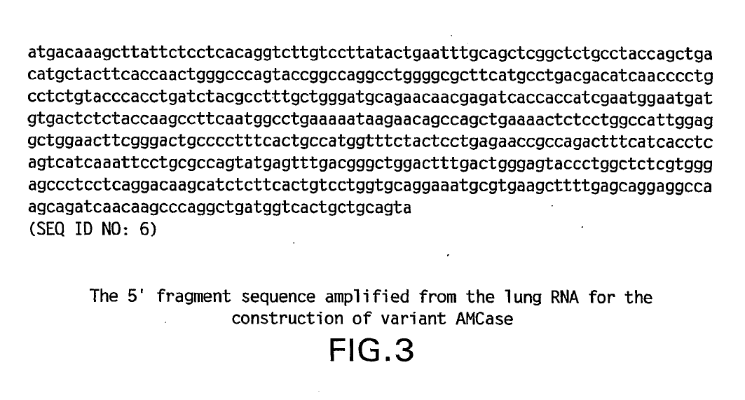

Two PCR amplification reactions were performed with Pfu Turbo® DNA polymerase (Stratagene Cat. #600153)) under the following conditions: 95° C. for 2 minutes and 30 cycles of 95° C. for 45 seconds, 55° C. for 45 seconds and 70° C. for 2 minutes. The reaction was terminated with one 10 minute cycle at 70° C. The PCR reactions were pooled and cloned in pPCR-Script Amp SK (+) vector (Stratagene Cat. #211188), and several clones were sequenced. We obtained a cDNA consisting of 561 bp (SEQ ID NO: 6) (FIG. 3).

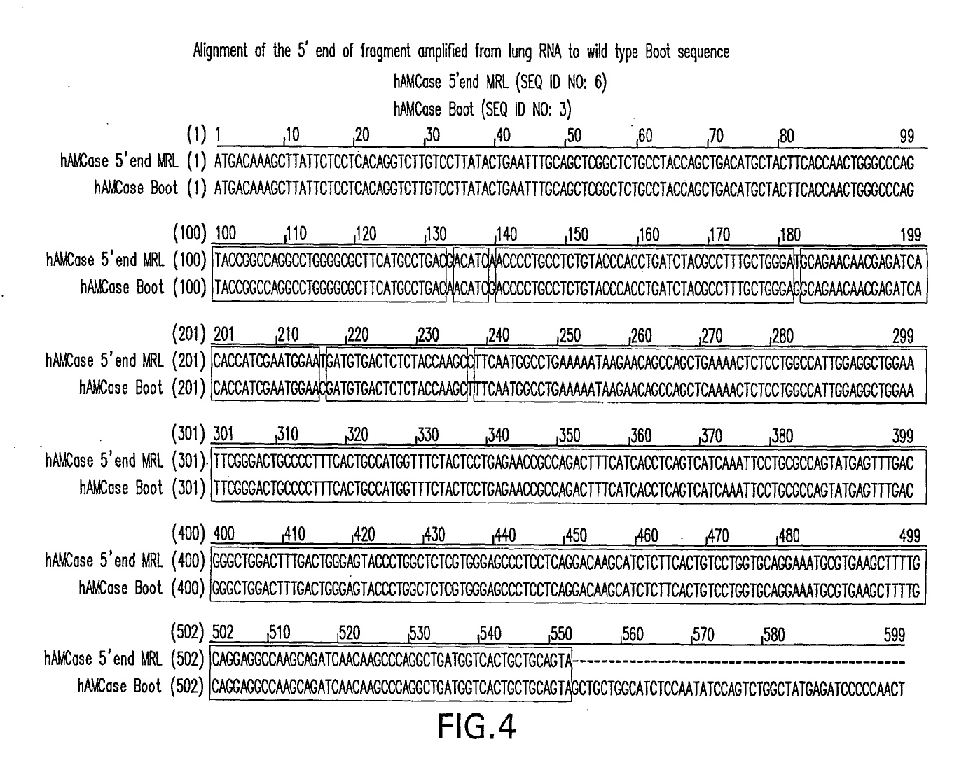

FIG. 4 provides an alignment comparing SEQ ID NO: 6 to the corresponding region of the wild-type “Boot” sequence (accession number AF 290004). There are five (5) base changes in the nucleotide sequence: 133G>A, 139 A>G, 182 T>G, 216 C>T and 237 T>C. The first three changes resulted in three amino acid changes: 45 N>D, 47 D>N and 61 R>M. The changes at 216 and 237 are silent mutations.

Generation of Full Length h-AMCase cDNA (MRL)

To obtain a full length h-AMCase, stomach (SEQ ID NO: 5) and lung (SEQ ID NO: 6) cDNAs were cut with the restriction enzyme Bal 1(Promega Cat. # R6691). A 281 bp fragment containing the five single point mutations was obtained from SEQ ID NO: 6 (lung cDNA clone). Bal I digestion of the stomach cDNA clone generated a 1190 bp fragment which was identical to the corresponding fragment of the wild-type clone (accession number AF 290004).

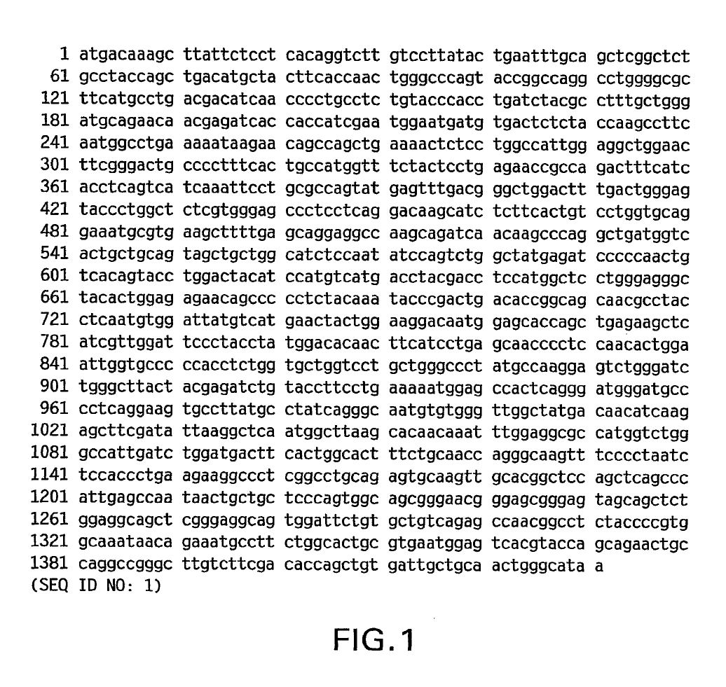

Ligation of these two fragments led to a full length cDNA for the variant AMCase (SEQ ID NO: 1) of the invention (FIG. 1). The resulting full length variant was cloned into the pcDNA™3.1 (In Vitrogen Cat. # V790) expression vector. The plasmid was used for transient transfection of HEK 293T and COS-7 cells.

### Example 2

### Alignment of Human AMCase Isoforms

Sequencing of the polynucleotide set forth in SEQ ID NO: 1, and analysis of an alignment performed using BLAST revealed that the cloned 5′ lung fragment contained five point mutations relative to the reference sequence. The five point mutations confer three amino acid substitutions relative to the wild-type isoform (AF 290004).

More specifically, the 5 point mutations found at the DNA level are 133A->G; 139G->A; 182G->T; 216C->T; and 237T->C. The number's relate to the nucleotide position and the first nucleotide before the arrow is present in the wild type AMCase and the nucleotide after the arrow represents the base found in the variant. FIG. 4 provides an alignment of SEQ ID NO: 1 and the corresponding region of wild-type human AMCase (from SEQ ID NO: 3) cDNA sequences.

The BLAST program is readily accessible from the files stored in /blast/executable from the NCBI database managed by the National Library of Medicine, National Institutes of Health (Bethesda, Md. USA) by using an FTP server. The details about the operation method are described in the installation and procedures packet available on the website for the National Institutes of Health (Bethesda, Md., USA).

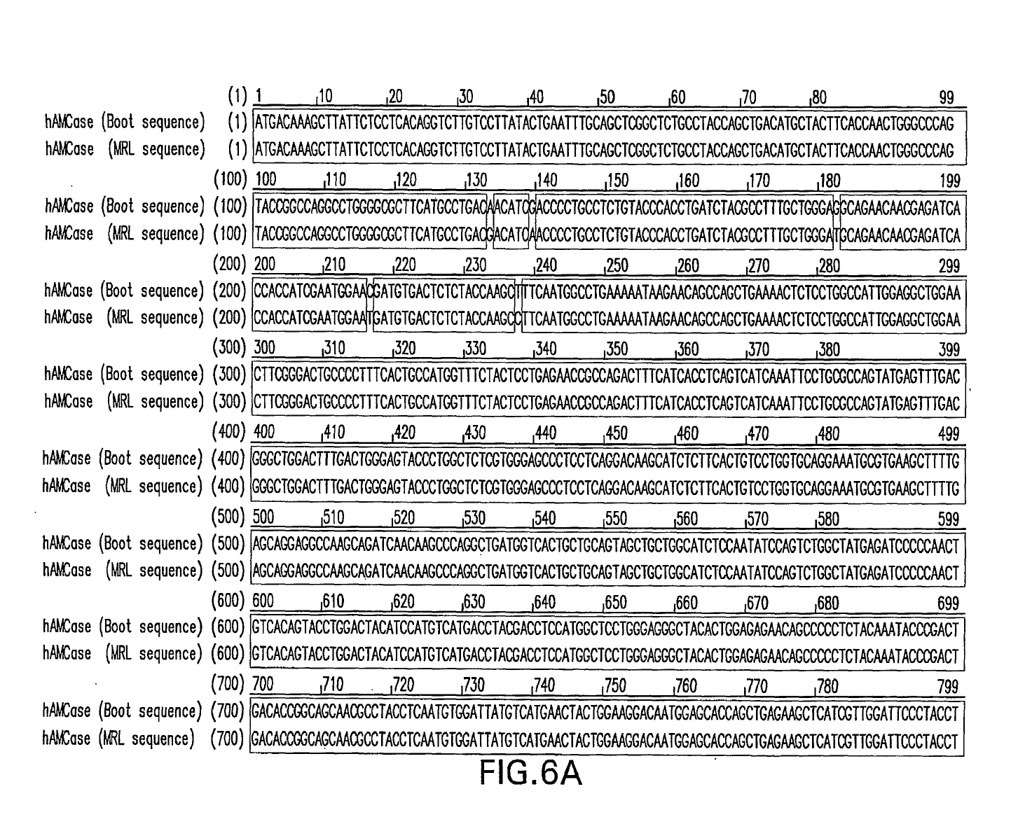

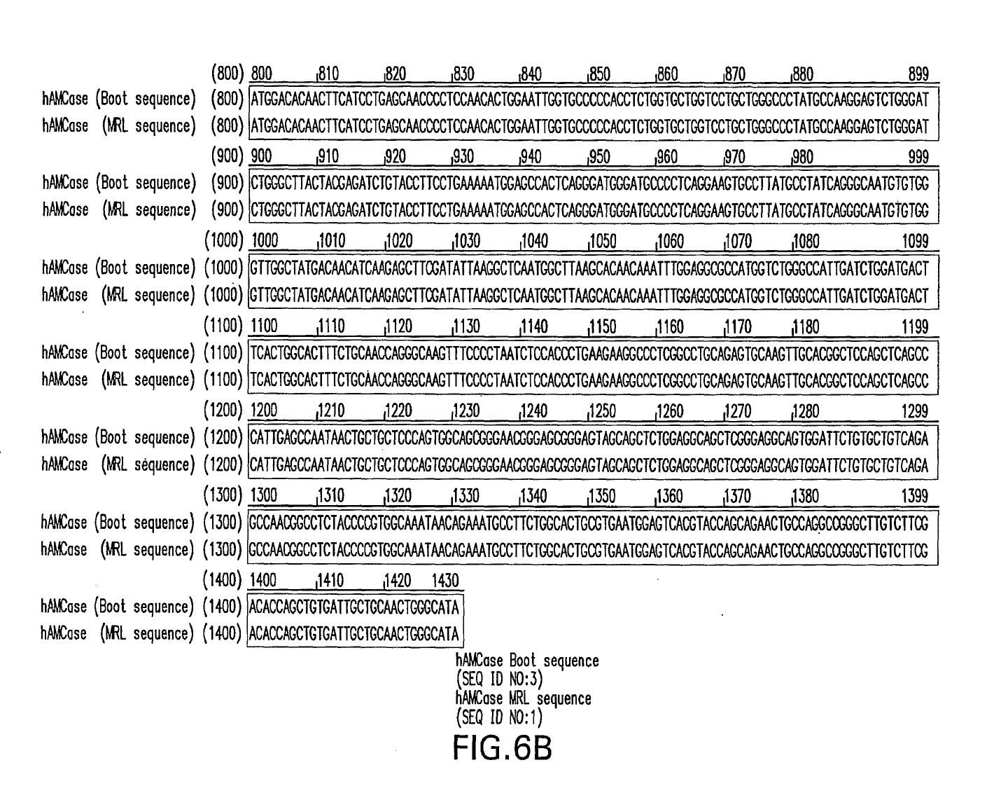

FIGS. 6A and 6B provide an alignment comparing the nucleotide sequence of SEQ ID NO: 1 with the nucleotide sequence encoding the wild-type AMCase (SEQ ID NO:3).

As noted above, as a consequence of these point mutations, the variant AMCase polypeptide set forth in SEQ ID NO: 2 comprises three different amino acid residues at positions 45, 47 and 61 (D45N, N47D, and M61R). FIG. 5 provides the amino acid sequences of human AMCase variant (SEQ ID NO: 2) of the invention.

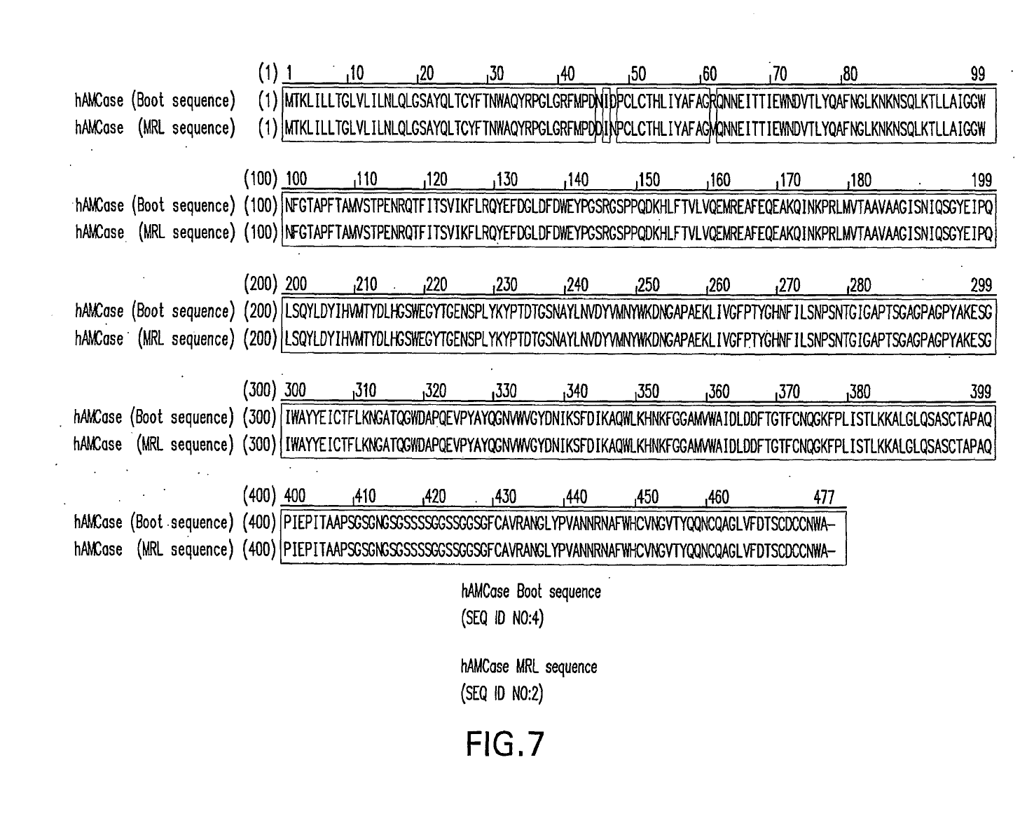

FIG. 7 provides an alignment comparing the amino acid sequence of SEQ ID NO: 2 (h-AMCase) (MRL sequence) with the amino acid sequence (SEQ ID NO: 4) (Boot sequence) of the wild-type AMCase product of SEQ ID NO: 3.

### Example 3

### Preparation of Wild-Type hAMCase (Boot) cDNA

Generation of Full Length Wild-Type h-AMCase (Boot)

Wild-type (i.e., Boot) hAMCase cDNA was used to produce recombinant wild-type protein (SEQ ID NO: 4) for use as a control in the characterization of the isoforms.

To obtain a cDNA encoding the wild-type AMCase amino acid sequence (SEQ ID NO: 3), three mutations: 133 G>A, 139 A>G and 182 T>G were introduced into SEQ ID NO: 1 using the QuikChange® Multi Site-Directed Mutagenesis kit (Stratagene Cat. # 200513). Clones were sequenced to confirm the mutagenesis. The wild-typed full length cDNA was cloned in the pcDNA™3.1 (InVitrogen Cat. # V790) expression vector. The plasmid was used in the transient transfections in HEK 293T and COS-7 cells.

### Example 4

### Expression and Purification of AMCase Enzymes

To facilitate cloning the full length cDNAs in the EcoRI and BamHI sites of the C-terminal p3xFLAG-CMV™-13 (Sigma Cat. #E 4776) expression vector, the following PCR primers were used to introduce cloning sites and for the elimination of the stop codon: AMCFLAF 5′-ataccgaattcgccaccatgacaaagcttattctcctc (SEQ ID NO: 17) and AMCFLAR 5′-tgcatggatcctgcccagttgeagcaatcacagctg (SEQ ID NO: 18). The vector contains the strong human cytomegalovirus (CMV) promoter, the SV40 replication origin and the preprotrypsin (PPT) leader sequence.

Plasmids encoding variant and wild-type h-AMCase or FLAG-tagged variant and wild-type hAMCase were transiently transfected into HEK-293T or COS-7 cells using the Lipofectamine™ 2000 transfection sreagent (Vitrogen Cat. # 11668-027). Transfected cells were kept in Opti-MEM® I low-serum media (Invitrogen Cat. #51985-034) for 3-6 days after transfection. Supernatants were harvested and chitinase activity was determined in the supernatants to assess the optimal conditions for maximum expression.

FLAG-tagged proteins were purified over an anti-FLAG M2 gel affinity column and eluted with a 3XFLAG peptide according to the manufacturers instructions Sigma chemical Co. Cat # A2220

Inhibitor potencies were determined for the purified proteins using a chitinase assay, such as the assay described below in Example 5.

### Example 5

### Enzymatic Activity of Variant and Wild-type AMCase

One of skill in the art will readily appreciate that alternative assays conducted using a protocol or substrate which differs from the assay described herein can readily be interchanged with this example.

Briefly, the assay consisted of fluorogenic substrate 4-methylumbelliferyl-β-D-N,N′-diacetylchitobiose (4MU-GlcNAc2; Sigma) at a final concentration of 22 μM, along with 1 nM of enzyme in a final volume of 100 μl. The fluorescence was read using excitation and emission wavelengths of 355 nm and 460 nm respectively.

More specifically the assay was performed in 96 well Costar poly plate (cat #3365.) The AMCase samples are introduced as 10 ul/wel samples of titered AMCase Supernatant (hAMCase variant or wild-type AMCase) or purified variant AMCase in pH 5.2 buffer. The samples are added to assay wells containing 90 ul/well of 22 uM substrate buffer (pH 5.2). A suitable substrate buffer, is 100 mM citric acid, 200 mM sodium phosphate buffer and sodium azide. Substrate solution (e.g., 55 ul of 2 mM substrate solution) (for example 5 mg 4-methylubelliferyl β-D-N,N′,N″-triacetylchitotriose (Sigma M5639) in 3.181 ml DMSO to give 2 mM solution) is added to each assay well. Incubate for 30 min at 30 C. Add 10 ul of reaction to 200 ul stop buffer (500 mM of sodium bicarbonate pH10.7) in Immulon 4 plate (Thermo electric corporation; cat #3855). Incubate 120 min at RT. Read plate in Victor 2 at EX 355 and EM 460 nm for 1 second.

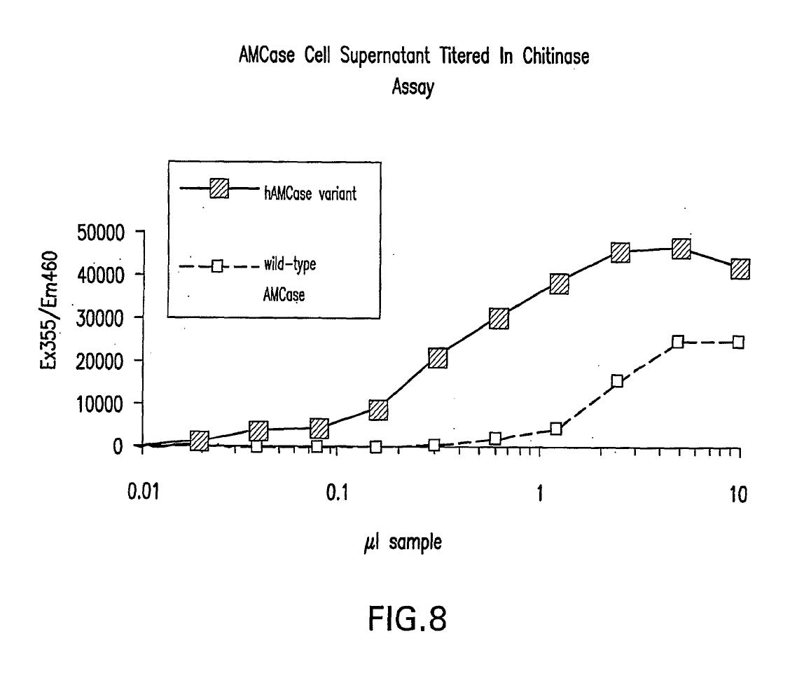

FIG. 8 is a graphic representation comparing the enzymatic activity of the hAMCase variant of the invention compared with the enzymatic activity of the wild-type AMCase (Boot AMCase).

### Example 6

### pH Profile and Reaction Max of the Variant and Wild-Type AMCases

In a variation of the activity assay described in example 5, the substrate buffer had different pH's 1.0, 2.0, 3.0, 4.0 and 5.2 to determine the pH optima of the proteins. The pH optima of the protein is not altered due to changes and is ˜3.0, which is consistent with it being an acidic mammalian chitinase.

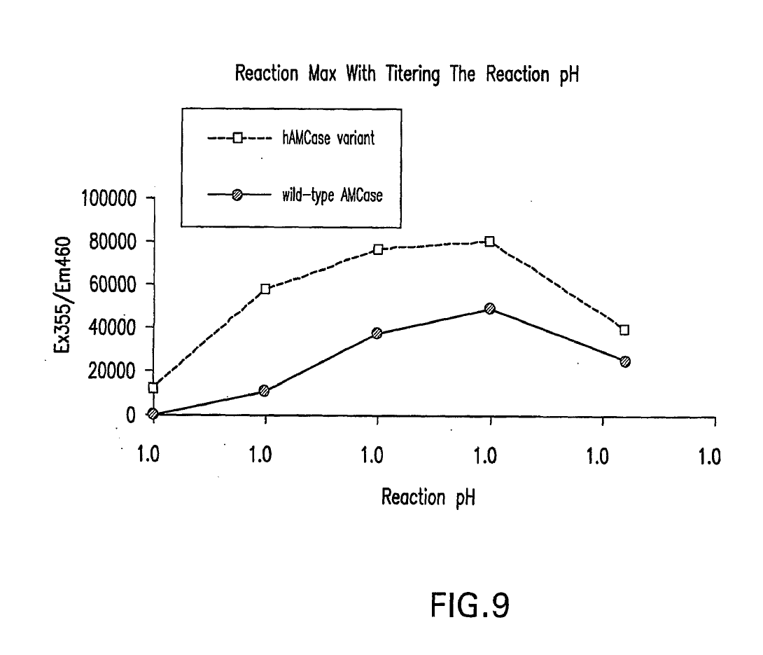

The data provided in FIG. 9 indicates that the variant enzyme does not show any changes in the pH optima relative to the wild-type enzyme.

### Example 7

### Allosamadin IC₅₀s at Different Reaction pHs

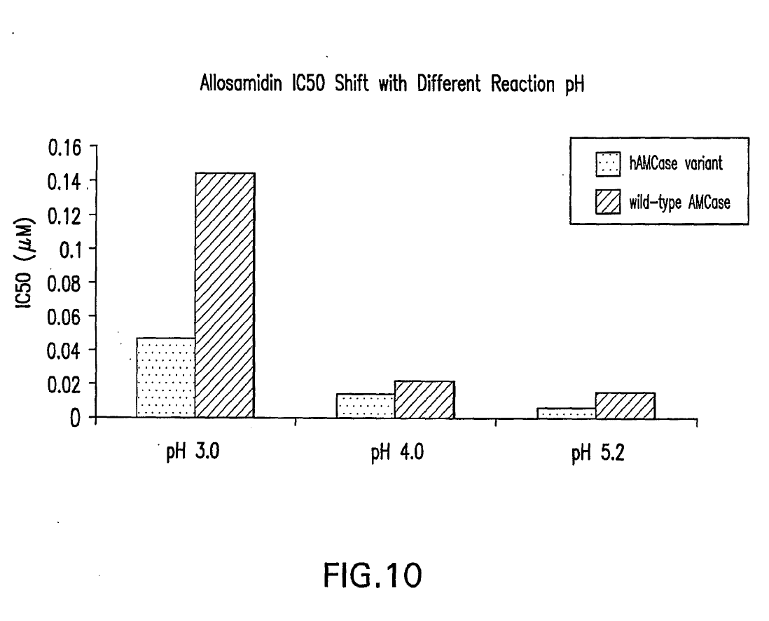

In another variation of the activity assay described in example 5, 1 ul of allosamidin (various concentrations generated by serial dilution) were added before the addition of the enzyme for IC50 determinations. Allosamidin was used to benchmark the AMCase isoforms. If the enzymatic activity of the variant protein was different from the activity of the wild-type enzyme then the IC 50 of the inhibitor would have changed. The data presented in FIG. 10 indicates that the variant isoform and the wildtype enzyme have similar IC50's for Allosamidin

### Example 8

### Variant AMCase Occurs in the General Population

To determine whether the nucleotide changes observed in the variant AMCase gene disclosed and claimed herein occur in vivo, a SNP (single nucleotide polymorphism) assay was designed to detect these changes at the genomic level. Genomic DNA were prepared from peripheral blood from subjects working at Merck in Rahway. All subjects signed a legal consent form. 200 □L of blood was used to isolate genomic DNA using QIAamp DNA blood mini kits Cat #51104. The assay was performed using the Custom TaqMan SNP Genotyping Assay (Applied Biosystems, product #4331349).

Two primers: Forward primer: TGAGGCACAGGGAGGGAAA (SEQ ID NO: 19), Reverse primer: CTGGTTTAGGATCCACTGAAGTGT (SEQ ID NO: 20) were synthesized to PCR amplify the sequence containing the SNP of interest. In addition, two probes labeled at the 5′ end with either a VIC® or 6-FAM™ reporter dye were designed to distinguish between the two known alleles. The probes also contain a non-fluorescent quencher at the 3′ end in order to suppress fluorescence by the intact probes. The probe containing the allele for the wild-type AMcase (probe sequence: CAGAGGCAAGGCCAA) (SEQ ID NO: 21) was labeled with the VIC® dye, while the probe for the hAMCase variant (probe sequence: AGAGGCAGGGCCAA) (SEQ ID NO: 22) was labeled with the 6-FAM™ dye. During PCR, the probes anneal specifically to a complementary sequence. The AmpliTaq® Gold DNA polymerase, contained in the TaqMan® Universal PCR Master Mix (Applied Biosystems, product #4304437), cleaves only those probes that are hybridized to the target. The cleavage separates the reporter dye from the non-fluorescent quencher, resulting in increased fluorescence by the reporter. Mismatches between a probe and target reduce the efficiency of probe hybridization. Furthermore, AmpliTaq Gold DNA polymerase is more likely to displace the mismatched probe rather than cleave it to release reporter dye. Thus, the fluorescence signal generated by the PCR amplification indicates which alleles are present in the sample.

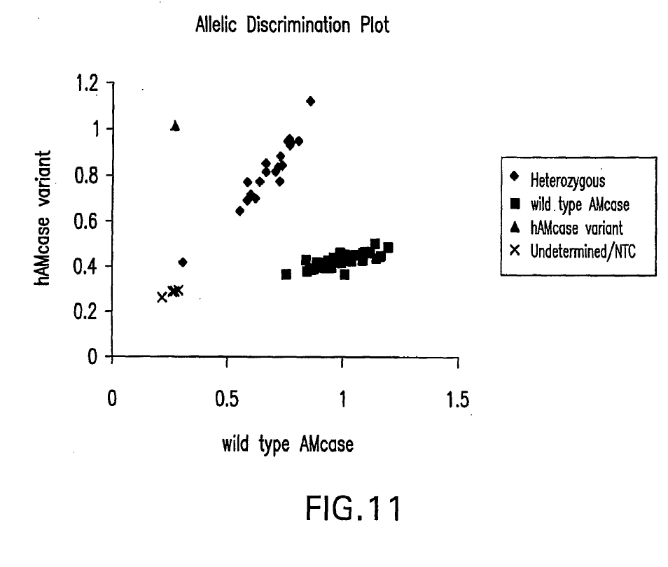

The results provided in Table 1 and FIG. 11 indicates that the AMCase variant sequence was detected amongst general human population. Out of the 56 individual DNA analysed, 1 subject was homozygous for AMCase variant and 21 were heterozygous for the wild type and variant AMCase sequences. These results indicate that the mutations reported in this application are present in the general population at the genomic DNA level.

TABLE 1

| Boot/MRL- — | # of Donors | Boot/MRL-TagMan | # of Donors |
|---|---|---|---|
| Homozygous Boot | 4 | Homozygous Boot | 44 |
| Homozygous MRL | 1 | Homozygous MRL | 1 |
| Heterozygous | 21 | Heterozygous | 21 |

### Example 9

### HTS Screening Assay for the Identification of AMCase Modulators

A HTS screening assay was carried out as a miniaturized version of the protocol described in Example 5. Specifically, the assay volumes consisted of 2 μL of assay buffer (100 mM Citric Acid, 200 mM Sodium Phosphate, 0.01% BSA, buffer pH −5.2.) The enzyme was diluted into the assay buffer to a final concentration of 2 nM. To this mixture 50mL of appropriate concentration compounds were added, followed by 2 μL of substrate buffer composed of 4-methylumbelliferyl β-D-N,N′,N″-triacetylchitotrioside (Sigma Cat. # M5639) is dissolved in assay buffer at 44 μM (22 μM final concentration in the reaction). After 30 minutes the reaction was terminated by 500 mM Sodium Carbonate, pH −11.8. The fluorescence was read on the Viewlux (Exc.—380 nm, Em.—450 nm). This assay yielded many important lead inhibitors belonging to the classes 1,6-naphthyridin-5-one, 1,8-naphthyridines, iso-indole-1-one, 4-imidazo[1,2]pyridine, 2-oxo-tetradecahydro-1H-indeno[5,4] quinoline carboxyamides and methylxanthines. Table 2 summarizes IC₅₀ values obtained for some representative small molecule compounds and allosamidin.

TABLE 2

| Inhibitors | IC₅₀ |
|---|---|
| Allosamidin | 10 nM |
| MRL 593 | 39 nM |
| MRL 098 | 90 nM |
| MRL 122 | 75 nM |
| MRL564 | 560 nM |
| MRL 296 | 500 nM |
| MRL 405 | 200 nM |

TABLE 3

IC50 (uM)

| Compound | WT AMCase | Variant AMCase |
|---|---|---|
| MRL-593 | 0.043 | 0.057 |
| MRL-405 | 0.234 | 0.213 |
| MRL-098 | 0.052 | 0.070 |

TABLE 4

| | MRL | Boot |
|---|---|---|
| Compound | AMCase (μM) | AMCase (μM) |
| MRL-5772 | 4.755 | 3.627 |
| MRL-1098 | 0.070 | 0.052 |
| MRL-7405 | 0.213 | 0.234 |
| MRL-4593 | 0.057 | 0.043 |

The data presented in Tables 3 and 4 which compares the enzymatic activities of wild-type (Boot AMCase) and the variant AMCase of the invention (MRL AMCase), indicates that the substitutions present in the variant sequence do not alter the potency of the representative modulators.

### Example 10

### Homology Modeling

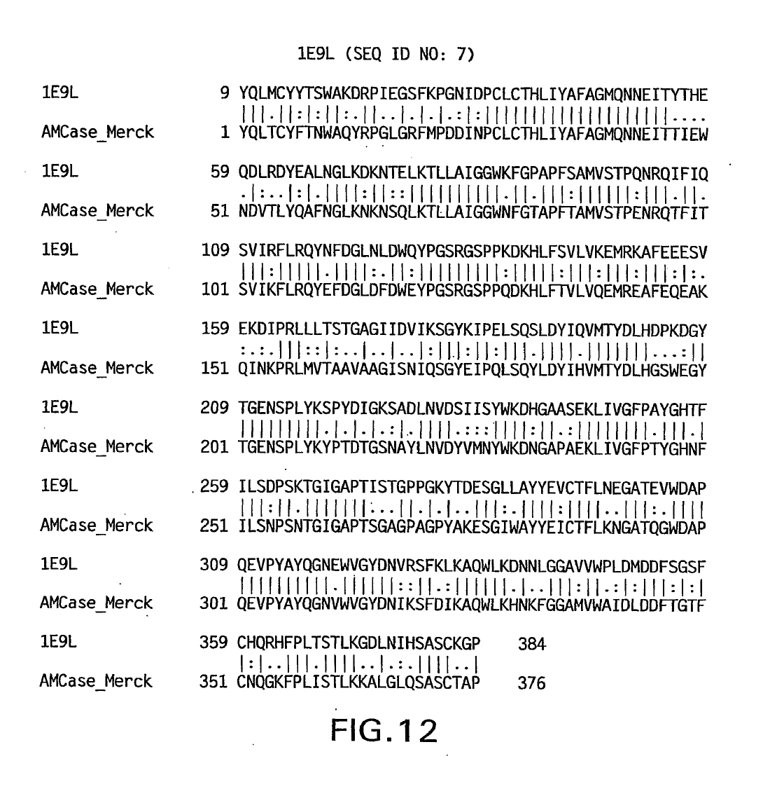

AMCase was modeled after a high resolution X-ray crystallography structure of its most homologous protein Ym1 (PDB code: 1E9L) using homology modeling function in the software MOE (Chemical Computing Group). Expected accuracy of the AMCase homology model is very high due to the high level of similarity between AMCase and template structure sequence. The (Smith-Waterman sequence alignment data is presented in FIG. 12.

AMCase homology model reveals that side chain of residue 61 points toward the inside of protein and is surrounded by hydrophobic residues, Ile66, Phe101, Phe106, Met109 and Phe 119. Water soluble globular proteins, such as AMCase, usually have an interior composed of non-polar hydrophobic amino acids with polar amino acid located on the surface of the molecule. This packing of hydrophobic residues is a consequence of hydrophobic effect, which is the most important factor that contributes to protein stability.

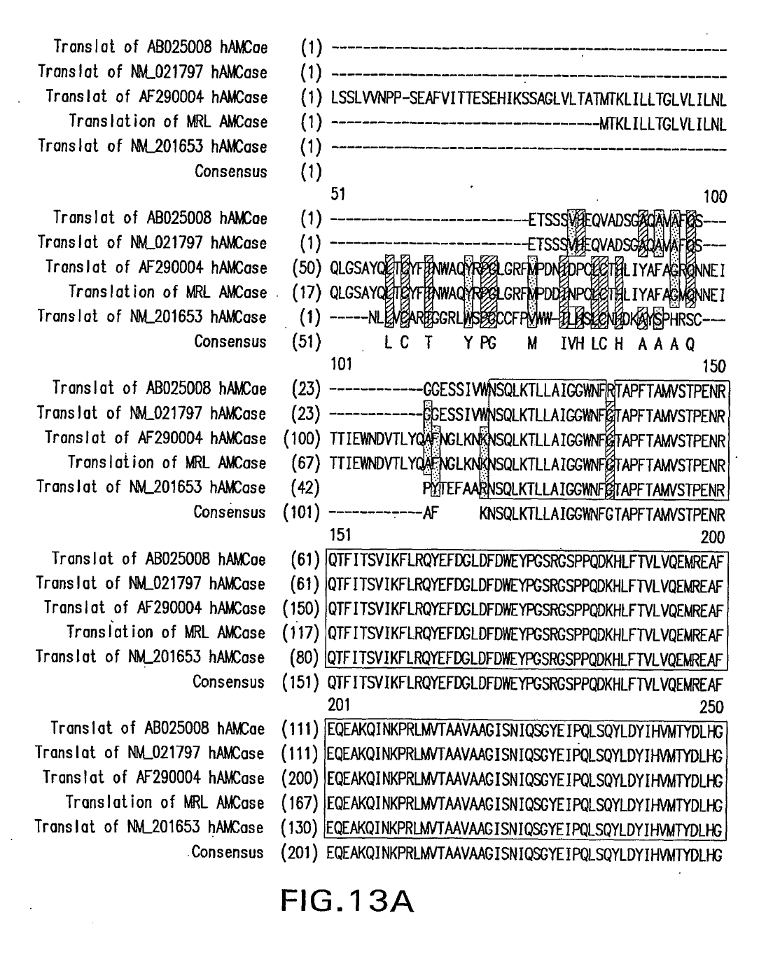

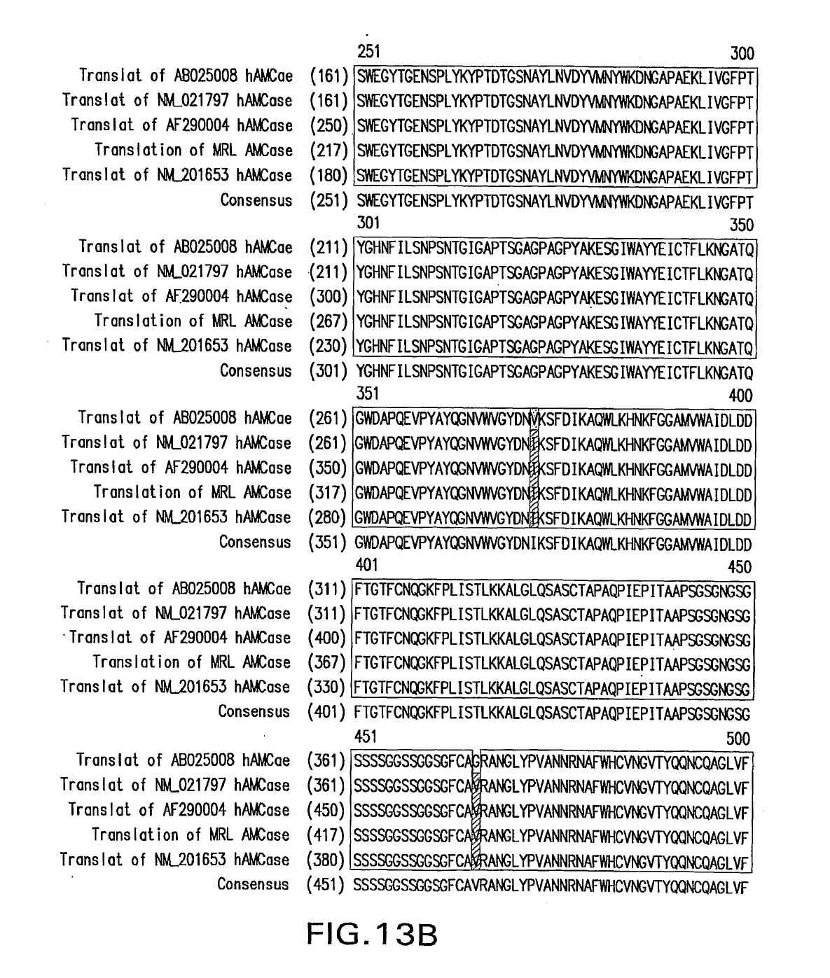

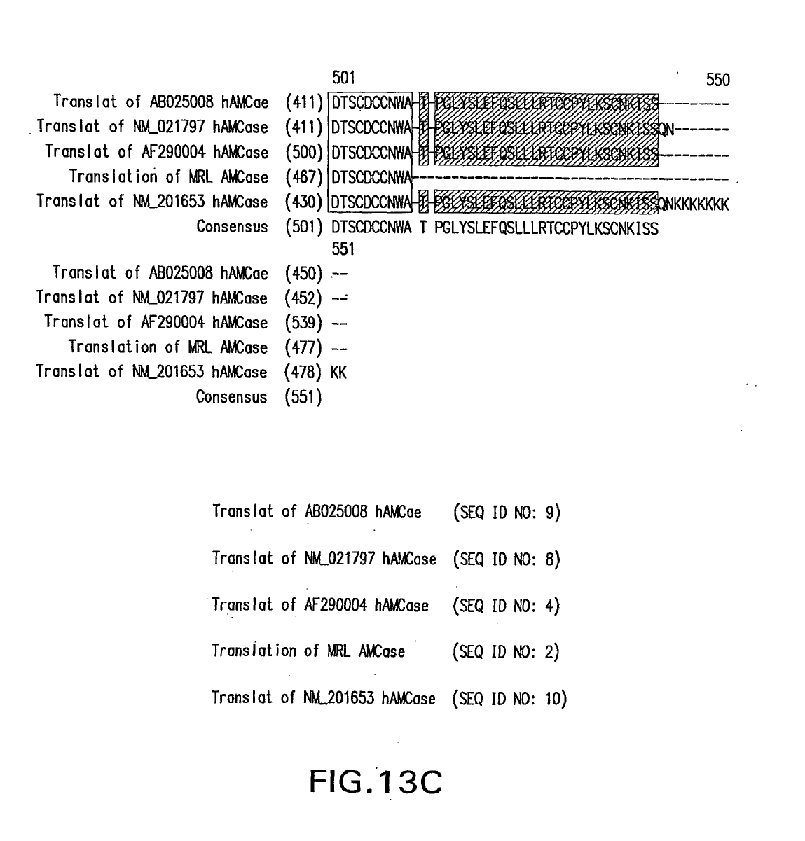

Therefore, in the residue 61 position which is at the end the second beta sheet of the TIM barrel of AMCase, a hydrophobic residue Met would provide a better stability to the structure than positively charged Arg by enhancing hydrophobic packing in the interior of the protein. Met61 could be replaced in that position by any hydrophobic residues such as Leu, Ile, Phe, Trp, Val or Cys leading to the desired hydrophobic effect akin to Met61. The sequence alignment provided in FIGS. 13A and 13B comparing the amino acid sequences of Elias (NM_f21797), Saito (AB025008), Boot (AF290004) (wild-type hAMCase, SEQ ID NO: 4) and the variant AMCase of the invention (SEQ ID NO: 2) reveals that the changes found in the MRL sequence are not represented in the other sequences reported to date in the literature.

In addition, two of the prior art sequences (Saito and Elias) lack the N-terminal 41 amino acids and have deletions of residues 98-113 in exon 3, making it unlikely for proper expression of the full length protein. Therefore based on the lack of N-terminal 41 residues and deletion of amino acid 98-113 (as reported by ELIAS and Saito) it would be difficult to predict the stability of the encoded proteins by these sequences.

Based on the disclosure provided herein, it is well within the skill of an investigator to identify other suitable assays for the use of the AMCase variant disclosed herein as druggable target for the development of compounds capable of modulating the enzymatic activity of this novel isoform of human acidic mammalian chitinase.

Other embodiments are within the scope of the following claims. A skilled artisan reading this disclosure will readily appreciate that various modifications may be made to the above-described examples without departing from the scope of the present invention

### REFERENCES

R. G. Boot, G. H. Renkema, A. Strijland, A. J. van Zonneveld, and J. M. F. G. Aerts, Cloning of a cDNA-encoding chitotriosidase, a human chitinase produced by macrophages, J. Biol. Chem., 270, 26252-26256 (1995).
R. G. Boot, E. F. C. Blommaart, E. Swart, K. Ghauharali van der Vlugt, N. Bijl, C. Moe, A. Place, and J. M. F. G. Aerts, Identification of a novel acidic mammalian chitinase distinct from chitotriosidase, J. Biol. Chem., 276, 6770-6778 (2001).
Zhou Zhu, Tao Zheng, Robert J. Horner, Yoon-Keun Kim, Ning Yuan Chen, Lauren Cohn, Qutayba Hamid, and Jack A. Elias, Acidic Mammalian Chitinase in Asthmatic Th2 Inflammation and IL-13 Pathway Activation, Science 11 Jun. 2004: 1678-1682.
Rolf G. Boot, Edward F. C. Blommaart, Erwin Swart, Karen Ghauharali-van der Vlugt, Nora Biji, Cassandra Moe, Allen Place, and Johannes M. F. G. Aerts, Identification of a Novel Acidic Mammalian Chitinase Distinct from Chitotriosidase, J. Biol. Chem., February 2001; 276: 6770-6778.
Akihiko Saitoa, Kouichi Ozakia, Tsutomu Fujiwaraa, Yusuke Nakamurab and Akira Tanigami, Isolation and—23—mapping of a human lung-specific gene, TSA 1902, encoding a novel chitinase family member, Gene Volume 239, Issue 2, 1 Nov. 1999, Pages 325-331.

1. An isolated human acidic mammalian chitinase (AMCase) variant consisting of the amino acid sequence set forth in SEQ ID NO: 2.

2. An isolated human AMCase variant with improved stability relative to wild-type AMCase wherein the variant comprises a substitution at a position corresponding to one or more of residues N45, D47, and R61 of SEQ ID NO: 4.

3. (canceled)

4. The isolated human AMCase variant according to claim 2, wherein the variant comprises D45N, N47D, and M61R substitutions at the designation positions of SEQ ID NO: 4.

5. An isolated nucleic acid encoding a human acidic mammalian chitinase variant consisting of an amino acid sequence selected from SEQ ID NO: 2 or SEQ ID NO: 4.

6. The isolated nucleic acid according to claim 4 wherein the nucleotide sequence set forth in SEQ ID NO: 1.

7. An expression vector comprising the nucleic acid encoding a human acidic mammalian chitinase variant consisting of an amino acid sequence selected from SEQ ID NO: 2 or SEQ ID NO: 4.

8. (canceled)

9. (canceled)

10. A host cell transformed with an expression vector comprising an isolated nucleic acid encoding a human acidic mammalian chitinase variant consisting of an amino acid sequence selected from SEQ ID NO: 2 or SEQ ID NO: 4.

11. (canceled)

12. A method for determining whether a test compound is capable of modulating AMCase enzymatic activity comprising:
(a) transfecting producer host cells with an expression vector comprising a nucleic acid encoding an AMCase variant selected from SEQ ID NO: 2 or SEQ ID NO: 4;
(b) culturing the transformed producer cells under conditions suitable to express the variant AMCase;
(c) purifying variant AMCase from the culture medium;
(d) exposing the variant AMCase to a test compound in the presence of a detectably labeled chitinase substrate;
(e) measuring the enzymatic activity of the variant AMCase;
(f) comparing the enzymatic activity of the variant AMCase observed in the absence of the test compound with the enzymatic activity observed in the presence of the compound; wherein if the level of enzymatic activity in the presence of the test compound differs from the amount of activity in the absence of the test compound, then the test compound is capable of modulating AMCase activity.

13. The method of claim 12 wherein expression vector comprises a nucleic acid sequence consisting of the nucleotide sequence set forth in SEQ ID NO: 1.

14. The method of claim 12 wherein the substrate in a N-acetylglucoseamine polymer which comprises β 1,4 glycosidic linkages.


SEQUENCE LISTING (23 sequences)

SEQ ID NO: 1 - DNA, 1431 bp, homo sapien
atgacaaagcttattctcctcacaggtcttgtccttatactgaatttgcagctcggctctgcctaccagctgacatgctacttcaccaactgggcccagtaccggccaggcctggggcgcttcatgcctgacgacatcaacccctgcctctgtacccacctgatctacgcctttgctgggatgcagaacaacgagatcaccaccatcgaatggaatgatgtgactctctaccaagccttcaatggcctgaaaaataagaacagccagctgaaaactctcctggccattggaggctggaacttcgggactgcccctttcactgccatggtttctactcctgagaaccgccagactttcatcacctcagtcatcaaattcctgcgccagtatgagtttgacgggctggactttgactgggagtaccctggctctcgtgggagccctcctcaggacaagcatctcttcactgtcctggtgcaggaaatgcgtgaagcttttgagcaggaggccaagcagatcaacaagcccaggctgatggtcactgctgcagtagctgctggcatctccaatatccagtctggctatgagatcccccaactgtcacagtacctggactacatccatgtcatgacctacgacctccatggctcctgggagggctacactggagagaacagccccctctacaaatacccgactgacaccggcagcaacgcctacctcaatgtggattatgtcatgaactactggaaggacaatggagcaccagctgagaagctcatcgttggattccctacctatggacacaacttcatcctgagcaacccctccaacactggaattggtgcccccacctctggtgctggtcctgctgggccctatgccaaggagtctgggatctgggcttactacgagatctgtaccttcctgaaaaatggagccactcagggatgggatgcccctcaggaagtgccttatgcctatcagggcaatgtgtgggttggctatgacaacatcaagagcttcgatattaaggctcaatggcttaagcacaacaaatttggaggcgccatggtctgggccattgatctggatgacttcactggcactttctgcaaccagggcaagtttcccctaatctccaccctgaagaaggccctcggcctgcagagtgcaagttgcacggctccagctcagcccattgagccaataactgctgctcccagtggcagcgggaacgggagcgggagtagcagctctggaggcagctcgggaggcagtggattctgtgctgtcagagccaacggcctctaccccgtggcaaataacagaaatgccttctggcactgcgtgaatggagtcacgtaccagcagaactgccaggccgggcttgtcttcgacaccagctgtgattgctgcaactgggcataa

SEQ ID NO: 2 - protein, 476 aa, homo sapien
MTKLILLTGLVLILNLQLGSAYQLTCYFTNWAQYRPGLGRFMPDDINPCLCTHLIYAFAGMQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWA

SEQ ID NO: 3 - DNA, 1430 bp, homo sapien
atgacaaagcttattctcctcacaggtcttgtccttatactgaatttgcagctcggctctgcctaccagctgacatgctacttcaccaactgggcccagtaccggccaggcctggggcgcttcatgcctgacaacatcgacccctgcctctgtacccacctgatctacgcctttgctgggaggcagaacaacgagatcaccaccatcgaatggaacgatgtgactctctaccaagctttcaatggcctgaaaaataagaacagccagctgaaaactctcctggccattggaggctggaacttcgggactgcccctttcactgccatggtttctactcctgagaaccgccagactttcatcacctcagtcatcaaattcctgcgccagtatgagtttgacgggctggactttgactgggagtaccctggctctcgtgggagccctcctcaggacaagcatctcttcactgtcctggtgcaggaaatgcgtgaagcttttgagcaggaggccaagcagatcaacaagcccaggctgatggtcactgctgcagtagctgctggcatctccaatatccagtctggctatgagatcccccaactgtcacagtacctggactacatccatgtcatgacctacgacctccatggctcctgggagggctacactggagagaacagccccctctacaaatacccgactgacaccggcagcaacgcctacctcaatgtggattatgtcatgaactactggaaggacaatggagcaccagctgagaagctcatcgttggattccctacctatggacacaacttcatcctgagcaacccctccaacactggaattggtgcccccacctctggtgctggtcctgctgggccctatgccaaggagtctgggatctgggcttactacgagatctgtaccttcctgaaaaatggagccactcagggatgggatgcccctcaggaagtgccttatgcctatcagggcaatgtgtgggttggctatgacaacatcaagagcttcgatattaaggctcaatggcttaagcacaacaaatttggaggcgccatggtctgggccattgatctggatgacttcactggcactttctgcaaccagggcaagtttcccctaatctccaccctgaagaaggccctcggcctgcagagtgcaagttgcacggctccagctcagcccattgagccaataactgctgctcccagtggcagcgggaacgggagcgggagtagcagctctggaggcagctcgggaggcagtggattctgtgctgtcagagccaacggcctctaccccgtggcaaataacagaaatgccttctggcactgcgtgaatggagtcacgtaccagcagaactgccaggccgggcttgtcttcgacaccagctgtgattgctgcaactgggcata

SEQ ID NO: 4 - protein, 476 aa, homo sapien [sequence omitted]

SEQ ID NO: 5 - DNA, 1229 bp, homo sapien
atgacaaagcttattctcctcacaggtcttgtccttatactgaatttgcagctcggaacagccagctgaaaactctcctggccattggaggctggaacttcgggactgcccctttcactgccatggtttctactcctgagaaccgccagactttcatcacctcagtcatcaaattcctgcgccagtatgagtttgacgggctggactttgactgggagtaccctggctctcgtgggagccctcctcaggacaagcatctcttcactgtcctggtgcaggaaatgcgtgaagcttttgagcaggaggccaagcagatcaacaagcccaggctgatggtcactgctgcagtagctgctggcatctccaatatccagtctggctatgagatcccccaactgtcacagtacctggactacatccatgtcatgacctacgacctccatggctcctgggagggctacactggagagaacagccccctctacaaatacccgactgacaccggcagcaacgcctacctcaatgtggattatgtcatgaactactggaaggacaatggagcaccagctgagaagctcatcgttggattccctacctatggacacaacttcatcctgagcaacccctccaacactggaattggtgcccccacctctggtgctggtcctgctgggccctatgccaaggagtctgggatctgggcttactacgagatctgtaccttcctgaaaaatggagccactcagggatgggatgcccctcaggaagtgccttatgcctatcagggcaatgtgtgggttggctatgacaacatcaagagcttcgatattaaggctcaatggcttaagcacaacaaatttggaggcgccatggtctgggccattgatctggatgacttcactggcactttctgcaaccagggcaagtttcccctaatctccaccctgaagaaggccctcggcctgcagagtgcaagttgcacggctccagctcagcccattgagccaataactgctgctcccagtggcagcgggaacgggagcgggagtagcagctctggaggcagctcgggaggcagtggattctgtgctgtcagagccaacggcctctaccccgtggcaaataacagaaatgccttctggcactgcgtgaatggagtcacgtaccagcagaactgccaggccgggcttgtcttcgacaccagctgtgattgctgcaactgggcataa

SEQ ID NO: 6 - DNA, 552 bp, homo sapien
atgacaaagcttattctcctcacaggtcttgtccttatactgaatttgcagctcggctctgcctaccagctgacatgctacttcaccaactgggcccagtaccggccaggcctggggcgcttcatgcctgacgacatcaacccctgcctctgtacccacctgatctacgcctttgctgggatgcagaacaacgagatcaccaccatcgaatggaatgatgtgactctctaccaagccttcaatggcctgaaaaataagaacagccagctgaaaactctcctggccattggaggctggaacttcgggactgcccctttcactgccatggtttctactcctgagaaccgccagactttcatcacctcagtcatcaaattcctgcgccagtatgagtttgacgggctggactttgactgggagtaccctggctctcgtgggagccctcctcaggacaagcatctcttcactgtcctggtgcaggaaatgcgtgaagcttttgagcaggaggccaagcagatcaacaagcccaggctgatggtcactgctgcagta

SEQ ID NO: 7 - protein, 376 aa, homo sapien
YQLMCYYTSWAKDRPIEGSFKPGNIDPCLCTHLIYAFAGMQNNEITYTHEQDLRDYEALNGLKDKNTELKTLLAIGGWKFGPASFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGP

SEQ ID NO: 8 - protein, 451 aa, homo sapien
ETSSSVHEQVADSGAQAVAFQSGGESSIVWNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNIKSFDIKAQWLKHNKFGGAMVWAIDLDDFTGTFCNQGKFPLISTLKKALGLQSASCTAPAQPIEPITAAPSGSGNGSGSSSSGGSSGGSGFCAVRANGLYPVANNRNAFWHCVNGVTYQQNCQAGLVFDTSCDCCNWATPGLYSLEFQSLLLRTCCPYLKSCNKISSQN

SEQ ID NO: 9 - protein, 449 aa, homo sapien
ETSSSVHEQVADSGAQAVAFQSGGESSIVWNSQLKTLLAIGGWNFRTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNVKSFDIKAQWLKHNKFGGAMVWAIDLDDFTGTFCNQGKFPLISTLKKALGLQSASCTAPAQPIEPITAAPSGSGNGSGSSSSGGSSGGSGFCAGRANGLYPVANNRNAFWHCVNGVTYQQNCQAGLVFDTSCDCCNWATPGLYSLEFQSLLLRTCCPYLKSCNKISS

SEQ ID NO: 10 - protein, 479 aa, homo sapien [sequence omitted]

SEQ ID NO: 11 - protein, 12 aa, Artificial Sequence
  peptide
FDGLDFDWEYPG

SEQ ID NO: 12 - protein, 12 aa, Artificial Sequence
  peptide
FDGLNLDWQFPG

SEQ ID NO: 13 - protein, 12 aa, Artificial Sequence
  peptide
FDGLDLAWLYPG

SEQ ID NO: 14 - DNA, 37 bp, Artificial Sequence
  primer
gccaccatgacaaagcttattctcctcacaggtcttg

SEQ ID NO: 15 - DNA, 37 bp, Artificial Sequence
  primer
ttatgcccagttgcagcaatcacagctggtgtcgaag

SEQ ID NO: 16 - DNA, 24 bp, Artificial Sequence
  primer
ggagatgccagcagctactgcagc

SEQ ID NO: 17 - DNA, 38 bp, Artificial Sequence
  primer
ataccgaattcgccaccatgacaaagcttattctcctc

SEQ ID NO: 18 - DNA, 36 bp, Artificial Sequence
  primer
tgcatggatcctgcccagttgcagcaatcacagctg

SEQ ID NO: 19 - DNA, 19 bp, Artificial Sequence
  primer
tgaggcacagggagggaaa

SEQ ID NO: 20 - DNA, 24 bp, Artificial Sequence
  primer
ctggtttaggatccactgaagtgt

SEQ ID NO: 21 - DNA, 15 bp, Artificial Sequence
  primer
cagaggcaaggccaa

SEQ ID NO: 22 - DNA, 14 bp, Artificial Sequence
  primer
agaggcagggccaa

SEQ ID NO: 23 - protein, 5 aa, Artificial Sequence
  motif
DXDXE